(19) United States
(12) Patent Application Publication
(10) Pub. No.: US 2018/0289845 A1
(43) Pub. Date: Oct. 11, 2018

(54) PREHEATED STERILIZING BOX FOR MEDICAL LABORATORY EQUIPMENT

(71) Applicant: Ka Yan CHAN, Hong Kong (HK)

(72) Inventor: Ka Yan CHAN, Hong Kong (HK)

(21) Appl. No.: 15/598,269

(22) Filed: May 17, 2017

(30) Foreign Application Priority Data
Apr. 7, 2017 (CN) 201710224868.3

(51) Int. Cl. A61L 2/00; A61L 2/20; A61L 2/04; A61L 2/10

(52) U.S. Cl. CPC A61L 2/0047; A61L 2/20; A61L 2/0005; A61L 2/10; A61L 2/04

(57) ABSTRACT

The present invention relates to a preheated sterilizing box for medical laboratory equipment, comprising a body, a cover hinged to the body through a hinge, and an attachment device for preventing collision between preheated equipment. The cover comprises a cover housing, a UV lamp disposed on the cover and a transparent cover board. The body is separated by the separator and the heat guiding plate into a first chamber for holding preheated sterilizing equipment, a second chamber disposed with temperature control circuitry, a third chamber disposed with a heating material and a fourth chamber disposed with a battery. An insulating layer is disposed on the inside wall of the first chamber and the third chamber, a temperature sensor is embedded in the insulating layer disposed on the inside wall of the first chamber. A number of heat guiding poles and through holes are disposed on the heat guiding plate. The attachment device is disposed on the through holes. The sterilizing box has a sensible structure, having the advantages of simple, convenient to use, well portable, can effectively solve the problem of secondary contamination during preheating and sterilizing of medical laboratory equipment.

### FIELD OF INVENTION

The present invention relates to the field of medical laboratory equipment, in particular to a preheated sterilizing box for medical laboratory equipment.

### BACKGROUND OF INVENTION

Medical laboratory is a field of using modern physical and chemical methods and means for clinical diagnosis, mainly researching on how to provide bases for clinical diagnosis through laboratory technology and medical equipment. During operation of medical testing, sterilization and preheating of testing equipment to be used is usually needed. Normal sterilizing boxes and heating boxes are large and not portable, therefore the usual method is to first sterilize the equipment in the sterilizing box, and then move to the heating box for preheating. This is not only tedious but also testing equipment may also be contaminated when moving from sterilizing box to the heating box, leading to unnecessary troubles. Furthermore, fragile equipment may collide with each other and break during sterilizing and continuous movement.

### SUMMARY OF INVENTION

The technical problem to be solved by the present invention is: to overcome the aforementioned problem, a preheated sterilizing box for medical laboratory equipment is provided. The sterilizing box has a sensible structure, having the advantages of simple, convenient to use, well portable, can effectively solve the problem of secondary contamination during preheating and sterilizing of medical laboratory equipment.

The technical solution used in the present invention to solve the technical problem is: a preheated sterilizing box for medical laboratory equipment, comprising a body, a cover hinged to the body through a hinge, and an attachment device for preventing collision between preheated equipment. The cover comprises a cover housing, a UV lamp disposed on the cover and a transparent cover board.

A separator and a heat guiding plate is mounted inside the body. The body is separated by the separator and the heat guiding plate into a first chamber for holding preheated sterilizing equipment, a second chamber, a third chamber and a fourth chamber. An insulating layer is disposed on the inside wall of the first chamber and the third chamber, a temperature sensor is embedded in the insulating layer disposed on the inside wall of the first chamber. A number of heat guiding poles extending towards the third chamber and a number of through holes are disposed on the heat guiding plate. The attachment device comprises a number of expansion screw buckles clipped onto the through hole, a rubber attachment strap and a connection ring, the connection ring is sleeved onto the expansion screw buckle, the two ends of the rubber attachment strap is attached to the connection ring respectively.

In a preferred embodiment, a fastening belt is disposed on the cover housing for connecting the cove and the body. One end of the fastening belt is fixed to the cover housing, a magnet is disposed at another end of the fastening belt.

In a preferred embodiment, the insulating layer is made of airgel.

In a preferred embodiment, the temperature sensor, temperature control circuitry disposed in the second chamber, heating material disposed inside the third chamber and a battery disposed inside the fourth chamber are all electrically connected with each other.

In a preferred embodiment, the heating material filled in the third chamber is carbon fiber heat tube.

The advantage of the present invention is: the preheated sterilizing box for medical laboratory equipment of the present invention, comprising a body, a cover hinged to the body and an attachment device. A UV lamp is disposed inside the cover, and a transparent cover board is disposed on the cover to prevent breaking of the UV lamp. The body is separated by the separator and the heat guiding plate into a first chamber for holding preheated sterilizing equipment, a second chamber having temperature control circuitry, a third chamber disposed with a heating material and a fourth chamber disposed with a battery. An insulating layer is disposed at an inside wall of the first chamber and the third chamber, and a temperature sensor is embedded in the insulating layer, allowing control of temperature based on needed preheat temperature. A number of heat guiding poles extending towards the third chamber and heat guiding holes are disposed on the heat guiding plate, increasing the area of contact with the material to be heated, increasing the utilization rate of heat. The battery disposed in the fourth chamber ensures the present invention to operate without interruption of power. The attachment device can be freely set up inside the body as needed, preventing equipment to be sterilized to collide and break. The sterilizing box has a sensible structure, having the advantages of simple, convenient to use, well portable, can effectively solve the problem of secondary contamination during preheating and sterilizing of medical laboratory equipment.

### BRIEF DESCRIPTION OF DRAWINGS

The present invention is further explained below with the accompanying drawings and embodiments.

FIG. 1 is an overall structural schematic diagram of a preheated sterilizing box for medical laboratory equipment according to the present invention.

FIG. 2 is a side cross-sectional schematic diagram of a mounting mechanism of a preheated sterilizing box for medical laboratory equipment according to the present invention.

Reference numbers in the drawings: 1 body, 11 first chamber, 12 second chamber, 13 third chamber, 14 fourth chamber, 15 insulation layer, 151 temperature sensor, 16 heat guiding plate, 161 heat guiding pole, 162 through hole, 17 separator, 2 cover, 21 hinge, 22 housing, 23 UV lamp, 24 transparent cover board, 25 fastening belt, 251 magnet, 3 attachment device, 31 expansion screw buckle, 32 rubber attachment strap, 33 connection ring.

### DETAILED DESCRIPTION OF INVENTION

The present invention is further explained below along with the accompanying drawings. The drawings are all simplified schematic diagrams which only illustrates the basic structure of the present invention, as such only the components material to the present invention are shown.

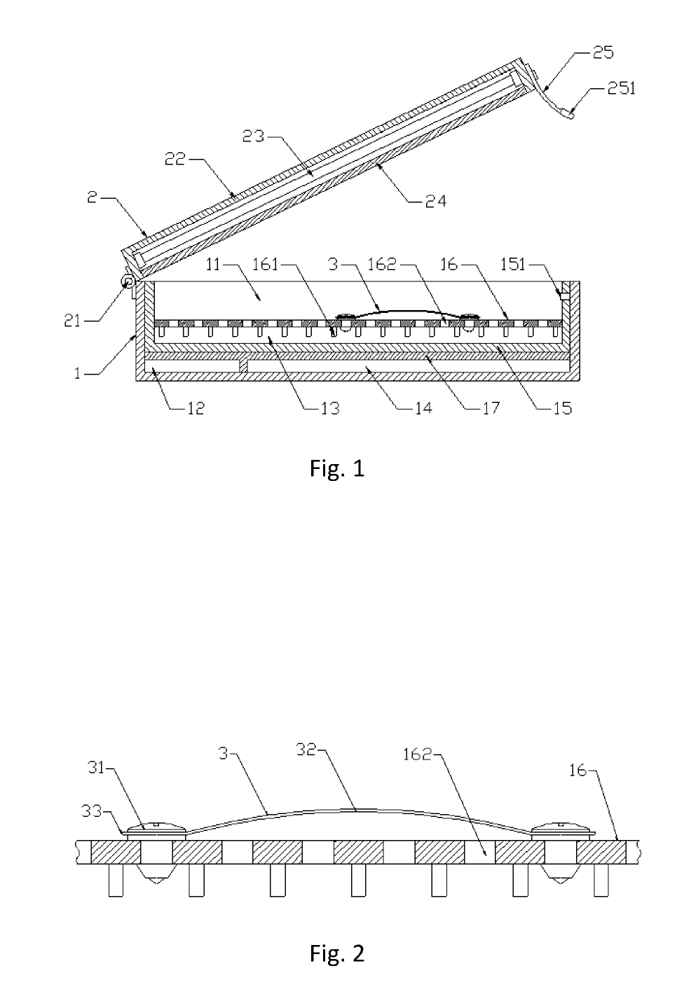

As shown in FIGS. 1-2, a preheated sterilizing box for medical laboratory equipment comprises a body 1, a cover 2 hinged to the body 1 through a hinge 21, and an attachment device 3 for preventing collision between preheated equipment. The cover 2 comprises a cover housing 22, a UV lamp 23 disposed on the cover and a transparent cover board 24.

A separator 17 and a heat guiding plate 16 is mounted inside the body. The body 1 is separated by the separator 17 and the heat guiding plate 16 into a first chamber 11 for holding preheated sterilizing equipment, a second chamber 12, a third chamber 13 and a fourth chamber 14. An insulating layer 15 is disposed on the inside wall of the first chamber 11 and the third chamber 13, a temperature sensor 151 is embedded in the insulating layer 15 disposed on the inside wall of the first chamber 11. A number of heat guiding poles 161 extending towards the third chamber 13 and a number of through holes 162 are disposed on the heat guiding plate 16. The attachment device 3 comprises a number of expansion screw buckles 31 clipped onto the through hole 162, a rubber attachment strap 32 and a connection ring 33, the connection ring 33 is sleeved onto the expansion screw buckle 31, the two ends of the rubber attachment strap 32 is attached to the connection ring 33 respectively.

A fastening belt 25 is disposed on the cover housing 22 for connecting the cover 2 and the body 1. One end of the fastening belt 25 is fixed to the cover housing 22, a magnet 251 is disposed at another end of the fastening belt 25. The insulating layer 15 is made of airgel. The temperature sensor 151, temperature control circuitry disposed in the second chamber 12, heating material disposed inside the third chamber 13 and a battery disposed inside the fourth chamber 14 are all electrically connected with each other. The heating material filled in the third chamber 13 is carbon fiber heat tube.

The preheated sterilizing box for medical laboratory equipment of the present invention, comprising a body, a cover hinged to the body and an attachment device. A UV lamp is disposed inside the cover, and a transparent cover board is disposed on the cover to prevent breaking of the UV lamp. The body is separated by the separator and the heat guiding plate into a first chamber for holding preheated sterilizing equipment, a second chamber having temperature control circuitry, a third chamber disposed with a heating material and a fourth chamber disposed with a battery. An insulating layer is disposed at an inside wall of the first chamber and the third chamber, and a temperature sensor is embedded in the insulating layer, allowing control of temperature based on needed preheat temperature. A number of heat guiding poles extending towards the third chamber and heat guiding holes are disposed on the heat guiding plate, increasing the area of contact with the material to be heated, increasing the utilization rate of heat. The battery disposed in the fourth chamber ensures the present invention to operate without interruption of power. The attachment device can be freely set up inside the body as needed, preventing equipment to be sterilized to collide and break. The sterilizing box has a sensible structure, having the advantages of simple, convenient to use, well portable, can effectively solve the problem of secondary contamination during preheating and sterilizing of medical laboratory equipment.

As revealed by the preferred embodiments of the present invention, based on the aforementioned description, a person skilled in the art can make various modifications and adjustments without departing from the technical concept of the present invention. The technical scope of the present invention is not limited to the specification but is defined by the scope of the claims.

What is claimed is:

1. A preheated sterilizing box for medical laboratory equipment, comprising a body, a cover hinged to the body through a hinge, and an attachment device for preventing collision between preheated equipment; the cover comprises a cover housing, a UV lamp disposed on the cover and a transparent cover board;
a separator and a heat guiding plate is mounted inside the body; the body is separated by the separator and the heat guiding plate into a first chamber for holding preheated sterilizing equipment, a second chamber, a third chamber and a fourth chamber; an insulating layer is disposed on the inside wall of the first chamber and the third chamber, a temperature sensor is embedded in the insulating layer disposed on the inside wall of the first chamber; a number of heat guiding poles extending towards the third chamber and a number of through holes are disposed on the heat guiding plate; the attachment device comprises a number of expansion screw buckles clipped onto the through hole, a rubber attachment strap and a connection ring, the connection ring is sleeved onto the expansion screw buckle, the two ends of the rubber attachment strap is attached to the connection ring respectively;
a fastening belt is disposed on the cover housing for connecting the cove and the body. One end of the fastening belt is fixed to the cover housing, a magnet is disposed at another end of the fastening belt.

2. The preheated sterilizing box for medical laboratory equipment according to claim 1, characterized in that: the insulating layer is made of airgel.

3. The preheated sterilizing box for medical laboratory equipment according to claim 1, characterized in that: the temperature sensor, temperature control circuitry disposed in the second chamber, heating material disposed inside the third chamber and a battery disposed inside the fourth chamber are all electrically connected with each other.

4. The preheated sterilizing box for medical laboratory equipment according to claim 1, characterized in that: the heating material filled in the third chamber is carbon fiber heat tube.
